(19) United States
(12) Patent Application Publication
(10) Pub. No.: US 2022/0115119 A1
(43) Pub. Date: Apr. 14, 2022

(54) MEDICAL DEVICE AND METHOD FOR SYNCHRONIZING CONFIGURATION INFORMATION THEREOF

(71) Applicant: SHENZHEN MINDRAY BIO-MEDICAL ELECTRONICS CO., LTD., Shenzhen (CN)

(72) Inventors: Yande HE, Shenzhen (CN); Shuaijun LIU, Shenzhen (CN)

(73) Assignee: SHENZHEN MINDRAY BIO-MEDICAL ELECTRONICS CO., LTD., Shenzhen (CN)

(21) Appl. No.: 17/499,791

(22) Filed: Oct. 12, 2021

(30) Foreign Application Priority Data
Oct. 12, 2020 (CN) 202011085681.8

(51) Int. Cl. G16H 40/40; H04L 29/08; H04W 4/029; G16H 40/67; A61B 90/90

(52) U.S. Cl. CPC G16H 40/40; H04L 67/12; A61B 2560/029; G16H 40/67; A61B 90/90; H04W 4/029

(57) ABSTRACT

The disclosure provides a medical device and a method for synchronizing configuration information of the medical device. The method includes acquiring configuration information from a terminal device and/or a remote control center as target configuration information, where the target configuration information includes at least one of communication authority configuration information, medical parameter configuration information and display configuration information, and the terminal device and/or the remote control center has at least one configuration information. The method also includes setting a corresponding configurable option of the medical device according to the target configuration information so as to synchronize the configuration information of the medical device as the target configuration information.

### CROSS-REFERENCE TO RELATED APPLICATIONS

This application claims benefits of priority to Chinese Patent Application No. 202011085681.8, filed Oct. 12, 2020, which is hereby incorporated by reference in its entirety.

### TECHNICAL FIELD

The disclosure relates to the field of medical devices, and more particularly to a medical device and a method for synchronizing configuration information thereof.

### BACKGROUND

After medical devices are installed or are installed after maintenance, a batch of medical devices generally needs to be configured to have the same interface or configuration parameters, or is required to have the same configuration parameters as those of similar medical devices installed previously. A current solution generally needs reconfiguration or manual input on the installed medical device. However, this approach is very inefficient and takes a long time if there are many medical devices needing to be configured.

### SUMMARY

According to a first aspect of the disclosure, a method for synchronizing configuration information of a medical device is provided, which includes acquiring configuration information from at least one of a terminal device and a remote control center as target configuration information, where the target configuration information includes at least one of communication authority configuration information, medical parameter configuration information and display configuration information, and at least one of the terminal device and the remote control center has at least one configuration information; and setting a corresponding configurable option of the medical device according to the target configuration information so as to synchronize the configuration information of the medical device as the target configuration information.

In some embodiments, the method includes receiving the configuration information that is broadcast by at least one of the terminal device and the remote control center via a network; and acquiring the target configuration information from the received configuration information.

In some embodiments, when the received configuration information includes a plurality of configuration information, one of the plurality of the received configuration information is acquired as the target configuration information; and when the received configuration information is unique configuration information, the unique configuration information is automatically acquired as the target configuration information.

In some embodiments, when the medical device is connected a network, the configuration information automatically recommended by at least one of the terminal device and the remote control center is received and presented so as to acquire the target configuration information.

In some embodiments, when the terminal device is a configured medical device and the medical device is directly connected to the configured medical device, the configuration information is directly acquired from the configured medical device as the target configuration information.

In some embodiments, the medical device accesses the remote control center via a network, and acquires the target configuration information from the remote control center.

In some embodiments, when the remote control center has a plurality of configuration information, one of the plurality of the configuration information is acquired as the target configuration information; and when the remote control center has unique configuration information, the unique configuration information is automatically acquired as the target configuration information.

In some embodiments, the at least one configuration information is identified by one or more of a device ID, a configuration information name, a user ID, a department, a ward number, a hospital bed number, and location information of the medical device, and the method further includes: acquiring the target configuration information from the at least one configuration information according to one or more of the device ID, the configuration information name, the user ID, the department, the ward number, the hospital bed number, and the location information of the medical device.

In some embodiments, the method further includes automatically acquiring, after the medical device is connected to the network, a latest version of configuration information in the configuration information that corresponds to the location information of the medical device as the target configuration information.

In another aspect, this disclosure provides a method for supplying configuration information for a medical device, which includes: receiving, by at least one of a terminal device and a remote control center, at least one configuration information for the medical device for storage; and supplying, by at least one of the terminal device and the remote control center, one of the at least one configuration information to the medical device as target configuration information, such that the medical device configures its corresponding configurable option according to the target configuration information to synchronize the configuration information of the medical device as the target configuration information, where the target configuration information includes at least one of communication authority configuration information, medical parameter configuration information and display configuration information.

In some embodiments, at least one of the terminal device and the remote control center receives configuration information adopted and automatically uploaded by a configured medical device for storage.

In some embodiments, at least one of the terminal device and the remote control center broadcasts the configuration information that at least one of the terminal device and the remote control center has via a network, such that the medical device receives and acquires the target configuration information therefrom.

In some embodiments, when it is detected that the medical device is connected to a network, at least one of the terminal device and the remote control center automatically recommends the configuration information to the medical device such that the medical device acquires the target configuration information therefrom.

In some embodiments, when the terminal device is a configured medical device and the configured medical device is directly connected to the medical device, the configured medical device directly transmits its configuration information to the medical device as the target configuration information.

In some embodiments, the remote control center presents the at least one configuration information when it is assessed by the medical device such that the medical device acquires the target configuration information therefrom.

In some embodiments, the at least one configuration information is identified by one or more of a device ID, a configuration information name, a user ID, a department, a ward number, a hospital bed number, and location information of the medical device, and the method further includes: supporting selection of the target configuration information according to one or more of the device ID, the configuration information name, the user ID, the department, the ward number, the hospital bed number and the location information of the medical device.

In some embodiments, after a user logs in the medical device, the configuration information associated with the user ID of the user is automatically transmitted to the medical device via the network such that the medical device acquires the target configuration information therefrom.

In some embodiments, the method further includes: automatically detecting the location information where the medical device is located; and automatically transmitting a latest version of configuration information in the configuration information that corresponds to the location information to the medical device via the network as the target configuration information.

In yet another aspect, this disclosure provides a medical device with configuration information to be synchronized. The medical device includes a configured option and a configurable option. The configurable option is set according to target configuration information to synchronize the configuration information of the medical device as the target configuration information. The target configuration information is configuration information that is acquired by the medical device from at least one of a terminal device and a remote control center and is comprised of at least one of communication authority configuration information, medical parameter configuration information and display configuration information, and at least one of the terminal device and the remote control center has at least one configuration information.

In some embodiments, the target configuration information is acquired from the configuration information received by means of network broadcast from at least one of the terminal device and the remote control center.

In some embodiments, when the received configuration information includes a plurality of configuration information, the medical device acquires one of the plurality of the configuration information as the target configuration information; and when the received configuration information is unique configuration information, the medical device automatically acquires the unique configuration information as the target configuration information.

In some embodiments, when the medical device is connected to a network, the medical device is capable of receiving and presenting the configuration information automatically recommended by at least one of the terminal device and the remote control center so as to acquire the target configuration information.

In some embodiments, when the terminal device is a configured medical device and the medical device is directly connected to the configured medical device, the medical device is capable of directly acquiring the configuration information of the configured medical device as the target configuration information.

In some embodiments, the medical device is capable of acquiring, when accessing the remote control center via a network, the target configuration information from the remote control center.

In some embodiments, when the remote control center has a plurality of configuration information, the medical device acquires one of the plurality of configuration information as the target configuration information; and when the remote control center has unique configuration information, the medical device automatically acquires the unique configuration information as the target configuration information.

In some embodiments, the at least one configuration information is identified by one or more of a device ID, a configuration information name, a user ID, a department, a ward number, a hospital bed number and location information of the medical device, the medical device is capable of acquiring one of the at least one configuration information as the target configuration information according to one or more of the device ID, the configuration information name, the user ID, the department, the ward number, the hospital bed number, and the location information of the medical device.

In some embodiments, the medical device is capable of automatically acquiring the configuration information associated with the user ID of a user via a network to acquire the target configuration information therefrom.

In some embodiments, when the acquired configuration information associated with the user ID of the user is unique configuration information, the medical device is capable of automatically acquiring the unique configuration information as the target configuration information.

In some embodiments, the medical device is capable of automatically acquiring, after being connected to a network, a latest version of configuration information in the configuration information that corresponds to the location information of the medical device as the target configuration information.

In still another aspect, this disclosure provides a terminal device and/or a remote control center for supplying configuration information to a medical device, where the terminal device and/or the remote control center include(s) a memory to store at least one configuration information for the medical device. The terminal device and/or the remote control center is/are capable of supplying one of the at least one configuration information to the medical device as the target configuration information, and the medical device configures its corresponding configurable option according to the target configuration information so as to synchronize the configuration information of the medical device as the target configuration information, where the target configuration information includes at least one of communication authority configuration information, medical parameter configuration information and display configuration information.

In some embodiments, the at least one configuration information includes configuration information adopted and automatically uploaded by a configured medical device.

In some embodiments, the terminal device and/or the remote control center are/is capable of broadcasting, via a network, the configuration information that the terminal device and/or the remote control center has, so as to supply the target configuration information to the medical device.

In some embodiments, when it is detected that the medical device is connected to a network, the terminal device and/or the remote control center are/is capable of automatically recommending the configuration information to the medical device such that the medical device acquires the target configuration information therefrom.

In some embodiments, when the terminal device is a configured medical device and the configured medical device is directly connected to the medical device, the configured medical device is capable of directly transmitting its configuration information to the medical device as the target configuration information.

In some embodiments, the remote control center further includes a display device; when the remote control center is assessed by the medical device, the display device presents the at least one configuration information for the medical device to acquire the target configuration information therefrom.

In some embodiments, the at least one configuration information is identified by one or more of a device ID, a configuration information name, a user ID, a department, a ward number, a hospital bed number and location information of the medical device, such that the medical device is capable of acquiring one of the at least one configuration information as the target configuration information according to one or more of the device ID, the configuration information name, the user ID, the department, the ward number, the hospital bed number, and the location information of the medical device.

In some embodiments, after a user logs in the medical device, the terminal device and/or the remote control center is capable of automatically transmitting the configuration information associated with the user ID of the user via a network to the medical device such that the medical device acquires the target configuration information therefrom.

In some embodiments, the terminal device and/or the remote control center further include(s) a detection apparatus for automatically detecting the location information where the medical device is located, and the terminal device and/or the remote control center are/is capable of automatically transmitting, when detecting the location information where the medical device is located, a latest version of configuration information in the configuration information that corresponds to the location information to the medical device as the target configuration information.

According to yet still further aspect of the disclosure, a computer-readable medium is provided. The computer-readable medium includes computer-readable instructions, which, when executed by a processor, cause the processor to carry out the method as described above.

In the medical device and the method for synchronizing the configuration information of the medical device of embodiments of the disclosure, there is no need to repeat import and export operations of the configuration information, the operation process is simple and easy, the degree of automation is high, and the time expenditure is less.

### BRIEF DESCRIPTION OF THE DRAWINGS

The above and other objects, features and advantages of the disclosure will become more apparent from the description of embodiments of the disclosure in detail with reference to the accompanying drawings. The accompanying drawings, which are intended to provide a further understanding of embodiments of the disclosure and constitute a part of this specification, are intended to explain the disclosure together with the embodiments of the disclosure and not to limit the disclosure. In the drawings, like reference numerals generally indicate like elements or steps.

FIG. 1 shows a schematic structural block diagram of a monitor or a module assembly in accordance with an embodiment of the disclosure;

FIG. 2 shows a schematic structural block diagram of a medical device in accordance with an embodiment of the disclosure;

FIG. 3 shows a schematic structural diagram of a monitor networking system for use in a hospital in accordance with an embodiment of the disclosure;

FIG. 4 shows a schematic structural block diagram of a terminal device and/or a remote control center in accordance with an embodiment of the disclosure;

FIG. 5 is a flowchart illustrating the a method for synchronizing configuration information of a medical device in accordance with an embodiment of the disclosure; and

FIG. 6 is a flowchart illustrating a method for supplying configuration information for a medical device in accordance with an embodiment of the disclosure.

### DETAILED DESCRIPTIONS

In order to make the objects, technical solutions, and advantages of the disclosure more apparent, the example embodiments according to the disclosure will be described in detail below with reference to the accompanying drawings. Apparently, the embodiments described are merely some, rather than all, of the embodiments of the disclosure. It should be understood that the disclosure is not limited by the example embodiments described herein. All other embodiments derived by those skilled in the art without creative efforts on the basis of the embodiments of the disclosure described in the disclosure shall fall within the scope of protection of the disclosure.

A current method for synchronizing configuration information of a medical device needs to repeat import and export operations of the configuration information and thus takes a long time to set the configuration information for a plurality of medical devices. In order to solve the above problems, an embodiment of the disclosure provides a method for synchronizing configuration information of a medical device. The method includes: acquiring configuration information from a terminal device and/or a remote control center as target configuration information, where the target configuration information includes at least one of communication authority configuration information, medical parameter configuration information and display configuration information, and the terminal device and/or the remote control center has at least one configuration information; and setting a corresponding configurable option of the medical device according to the target configuration information so as to synchronize the configuration information of the medical device as the target configuration information.

The medical device and the method for synchronizing the configuration information of the medical device of embodiments of the disclosure do not need to repeat import and export operations, the operation process is simple and easy, the degree of automation is high, and the time expenditure is less.

The solution of the disclosure will be described in detail below with respect to specific embodiments.

A first embodiment provides a medical device 10 that needs synchronization in its configuration information. By way of example, the medical device 10 may be a monitor (e.g., a multi-parameter monitor), a ventilator, etc., which is not limited by the disclosure.

In an embodiment, the medical device 10 may include a monitor or a module assembly, and further include any one monitor or module assembly, such as a single-parameter monitor, a multi-parameter monitor, a vital sign monitor, a ward round monitor. The monitor or module assembly has at least one working mode in which physiological parameters of a monitored subject are measured. In different working modes, the medical device 10 may execute one or more different measurement functions to adapt to different measurement functions and measurement methods required by a user in different geographic areas, different scenarios, or different departments. For example, BP average and BMI (body mass index) measurements are often required in a Doctor office, but the BMI measurement is not required in a general ward, and CCHD measurement is required in a neonatal department, but is not required in an adult general ward. The user may configure the configuration information in those different working modes in a custom manner, thereby configuring different measurement functions in different working modes according to the requirements of the user to adapt to their measurement requirements in different scenarios or different departments. It may be understood that the configuration information in each working mode may also be default configuration information, and the user may select the default configuration information to configure the measurement functions in different working modes or may configure the measurement functions in different working modes in a custom manner, which is not limited herein. By setting different configuration information, the user may define physiological parameters acquired by means of a parameter measurement accessory as per required time intervals or number of times, such as body temperature and blood pressure; or may define physiological parameters, such as urine volume, consciousness, oxygen supply and blood glucose, that are manually entered by the user instead of being obtained by means of the parametric measurement accessory.

In an embodiment, the monitor includes at least two working modes that are set according to a scenario or a department used by the user. For example, the working modes in a general ward include at least one of an initial evaluation working mode, a ward round working mode and a continuous monitoring working mode, and the working modes in an emergency ward include at least one of a triage working mode and a point measurement working mode. The initial evaluation working mode is configured for initial evaluation on vital signs of a target just admitted to a hospital or a target in the emergency department, in which mode the physiological parameters of the target at admission are typically measured. The ward round working mode is suitable for monitoring the target single time or multiple times during hospitalization. The continuous monitoring working mode is suitable for continuously acquiring the physiological parameters of the target within a period of time and is used for continuously monitoring the target. In the continuous monitoring working mode, the physiological parameters are automatically acquired at a certain time interval of 1 second, 30 seconds, 1 minute, 5 minutes, 30 minutes, 6 hours, 12 hours, etc., which is not limited herein. The triage working mode is suitable for triage in the emergency department. In the point measurement working mode, the vital signs of the target at a certain moment may be recorded as required. In each working mode, the acquired configuration information such as the types of the physiological parameters, the number of times of physiological parameter measurement, the calculation method for the physiological parameters, the display mode of the physiological parameters and the alarm setting may each vary, may be determined by the default configuration information or may also be configured in a custom manner and modified by the user. The name of each working mode may be set by the user as required, which is not limited herein.

First, a schematic structure of the medical device 10 will be described below by taking a monitor as an example.

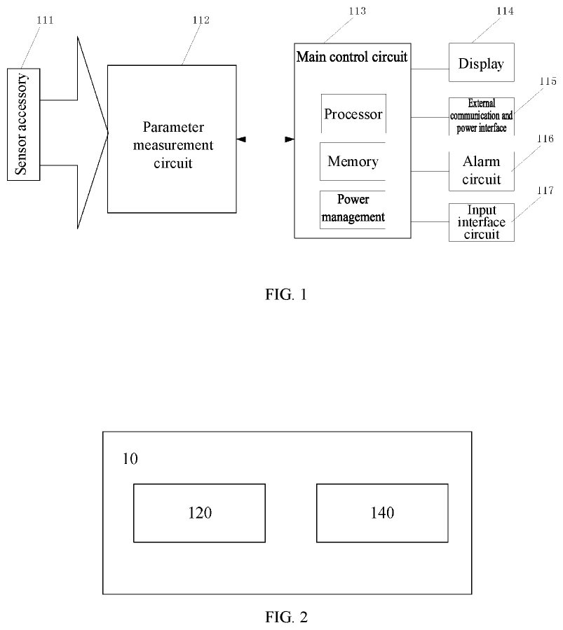

Referring to FIG. 1, FIG. 1 shows a schematic structural block diagram of a monitor or a module assembly in accordance with an embodiment of the disclosure. As shown in FIG. 1, the monitor or the module assembly may include at least a parameter measurement circuit 112. The parameter measurement circuit 112 may include at least the parameter measurement circuit 112 corresponding to a physiological parameter, and the parameter measurement circuit 112 may include at least one of an ECG signal parameter measurement circuit, a respiratory parameter measurement circuit, a body temperature parameter measurement circuit, a blood oxygen parameter measurement circuit, anon-invasive blood pressure parameter measurement circuit, an invasive blood pressure parameter measurement circuit, etc. Each parameter measurement circuit 112 is connected to an externally inserted sensor accessory 111 via a corresponding sensor interface. The sensor accessory 111 includes at least one of detection accessories corresponding to the detection of the physiological parameters such as ECG, respiration, blood oxygen, blood pressure and body temperature. The parameter measurement circuit 112 and the sensor accessory 111 above may be built-in parts of the monitor or the module assembly, or may be combined with or plugged in the monitor or the module assembly as a removable parameter measurement module or an accessory module. The parameter measurement circuit 112 is mainly configured to connect to the sensor accessory 111 to obtain collected physiological parameter signals, and may include at least two measurement circuits for the physiological parameters. The parameter measurement circuit 112 may be, but is not limited to, a physiological parameter measurement circuit 112 (module) or the sensor accessory 111 for collecting human physiological parameters, etc. Specifically, the parameter measurement circuit 112 obtains, by means of an extended interface, a physiological sampled signal related to a patient collected by an external physiological parameter sensor accessory, processes the physiological sampled signal and then obtains physiological data for alarming and display. The extended interface may be further configured to output a control signal that is output from a main control circuit 113 for collecting the physiological parameters to an external physiological parameter monitoring accessory through a corresponding interface, so as to control the monitoring of the physiological parameters of the patient.

The monitor or the module assembly may further include the main control circuit 113. The main control circuit 113 may include at least one processor and at least one memory. In other embodiments, the main control circuit 113 may further include at least one of a power management module, a power IP module and an interface conversion circuit, etc. The power management module is configured to control the power on and off of the whole device, a power-on sequence of each power domain inside a board card, battery charging and discharging, etc. The power IP module is a separate power module firmed by associating a principle diagram of a power supply circuit unit that is frequently and repeatedly invoked, with a PCB layout. That is, an input voltage is converted into an output voltage by means of a predetermined circuit, where the input voltage and the output voltage are different. For example, the voltage of 15 V is converted into 1.8 V, 3.3 V, 3.8 V, etc. It can be understood that the power IP module may be single-channel or multi-channel. If the power IP module is single-channel, the power IP module may convert one input voltage into one output voltage. If the power IP module is multi-channel, the power IP module may convert one input voltage into a plurality of output voltages, and voltage values of the plurality of output voltages may be the same or different, such that different voltage requirements of a plurality of electronic components can be met at the same time. In addition, the module has few external interfaces, and works in the system as a black box decoupled from an external hardware system, which improves the reliability of the entire power system. The interface conversion circuit is configured to convert a signal output by a master control minimum system module (i.e., at least one processor and at least one memory in the main control circuit 113) into an input standard signal required to be received by an actual external device. For example, supporting an external VGA display function is to convert an RGB digital signal output by a master control CPU into a VGA analog signal, and supporting an external network function is to convert an RMII signal into a standard network differential signal.

In addition, the monitor or the module assembly may also include one or more of a local display 114, an alarm circuit 116, an input interface circuit 117, and an external communication and power interface 115. The main control circuit 113 is configured to coordinate and control each board card, circuit and device in the multi-parameter monitor or the module assembly. In this embodiment, the main control circuit 113 is configured to control data interaction and control signal transmission between the parameter measurement circuit 112 and a communication interface circuit, and transmit the physiological data to the display 114 for display, or may receive a user control instruction entered from a touchscreen or a physical input interface such as a keyboard and keys, and may also output a control signal for collecting the physiological parameters. The alarm circuit 116 may be an audible and visual alarm circuit. The main control circuit 113 completes the calculation of the physiological parameters, and may send calculation results and waveforms of the parameters to the host (such as a host with a display 114, a PC, and a central station) by means of the external communication and power interface 115. The external communication and power interface 115 may be one or a combination of local area network interfaces composed of Ethernet, a token ring, a token bus, and an optical fiber distributed data interface (FDDI) as the backbone of these three networks, or may be one or a combination of wireless interfaces such as infrared, Bluetooth, wifi, and WMTS communication, or may be one or a combination of wired data connection interfaces such as RS232 and USB. The external communication and power interface 115 may also be one of a wireless data transmission interface and a wired data transmission interface or a combination thereof. The host may be any computing device, such as a host of the monitor, an electrocardiograph machine, an ultrasonic diagnosis instrument, and a computer, and can form a monitoring device once installed with matching software. The host may alternatively be a communication device such as a mobile phone, and the multi-parameter monitor or the module assembly sends, by means of a Bluetooth interface, data to the mobile phone supporting Bluetooth communication, so as to implement remote transmission of data.

The monitoring module assembly may be arranged outside a housing of the monitor as an independent externally inserted parameter module, may be inserted into the host (including a main control board) of the monitor to form a plug-in monitor as a part of the monitor, or may be connected to the host (including the main control board) of the monitor by means of a cable, and the externally inserted parameter module is used as an external accessory of the monitor. Alternatively, the parameter processing module may also be built in the housing to be integrated with a main control module, or physically separated and arranged in the housing to form an integrated monitor.

Referring to FIG. 2, the medical device 10 may include a configured option 120 and a configurable option 140, where if the medical device is a newly purchased device or a maintained device, etc., the configurable option 140 of the medical device may need to be set, and the configuration information of the device is synchronized to be the same as that of other devices.

By way of example, the configured option 120 may include some basic configuration items, such as baseline configuration items that have been set with basic configuration information. By way of example, the baseline configuration information includes configuration information such as various preset thresholds of the medical device, for example, an alarm threshold.

By way of example, the configurable option 140 may include a communication authority configuration item, a medical parameter configuration item, and/or a display configuration item, etc., which are not limited by the disclosure. The configurable option may be set according to the target configuration information to synchronize the configuration information of the medical device as the target configuration information.

The target configuration information may be the same configuration information as the configuration information of a medical device that has been set already, or may be any configuration information that the user desires.

By way of example, the target configuration information may include at least one of communication authority configuration information, medical parameter configuration information and display configuration information, etc. The medical parameter configuration information refers to configuration information of parameters required by the medical device in the process of completing each medical function, including algorithm configuration information, function sequence configuration information, etc. The algorithm configuration information may include an algorithm required to implement a certain medical function, such as algorithms to implement blood pressure measurement, blood oxygen measurement, body temperature measurement, EWS (Early Warning Scoring), a physiological parameter averaging algorithm, etc. The function sequence configuration information may include information of sequence and interval time of executing the algorithms or each medical function. For example, body temperature measurement is performed first, followed by blood pressure measurement second, and finally blood oxygen measurement is performed. As another example, the blood pressure is measured and is to be measured again after 5 minutes. The communication authority configuration information refers to the information that the medical device is connected to an internal network or a medical system of the hospital, which is generally implemented by means of an encryption certificate, such as WiFi encryption certificate (enterprise encryption certificate) and ssl encryption certificate. The display configuration information may include display area information and display style information, where the display area information may be information for limiting the configuration of different measurement functions in each area of a display interface. For example, a blood pressure measurement function area is arranged in an upper right position for prompting blood pressure measurement, inputting and displaying a blood pressure measurement result, and outputting all information related to a blood pressure measurement function, such as a blood pressure alarm. For example, a body temperature measurement value is shown in a lower left corner for prompting body temperature measurement, inputting and displaying a body temperature measurement result, and outputting all information related to a body temperature measurement function, such as a body temperature alarm. The display style information may include information for setting the font, the word size, the word color, the underline, the highlight, etc. of the displayed information. It should be understood that the target configuration information may also include other configuration information, which is not limited by the disclosure.

By way of example, the target configuration information may be configuration information acquired by the medical device 10 from a terminal device and/or a remote control center. The terminal device and/or the remote control center may have at least one configuration information. By way of example, the terminal device may be a medical device or a mobile storage device, etc. By way of example, the remote control center may include a server, a central station or a device management system, etc. By way of example, the at least one configuration information may be set by the user and then stored in the remote control center, or the user may store the configuration information adopted by the configured medical device in the remote control center, or the medical device may automatically upload the configuration information adopted by itself into the remote control center, or the at least one configuration information may be the configuration information locally stored and adopted by the medical device, etc. By way of example, the at least one configuration information may be stored in the form of a configuration information packet, etc. By way of example, the at least one configuration information may be identified by one or more of a device ID, a configuration information name, a user ID, a department, a ward number, a hospital bed number, and location information of the medical device.

By way of example, the target configuration information may be acquired from the configuration information received by means of network broadcast from the terminal device and/or the remote control center. Specifically, the terminal device and/or the remote control center broadcasts, via a network, the configuration information that the terminal device and/or the remote control center has, and the medical device receives the broadcast configuration information after connecting to the network and acquires the configuration information required by the medical device itself as the target configuration information. By way of example, if the received configuration information is a plurality of configuration information, the medical device 10 may acquire (e.g., selected by the user) one of the plurality of the configuration information as the target configuration information. By way of example, the medical device 10 may acquire (e.g., selected by the user) one of the received configuration information as the target configuration information according to one or more of the device ID, the configuration information name, the user ID, the department, the ward number, the hospital bed number, and the location information of the medical device. By way of example, if the received configuration information is unique configuration information, the medical device 10 may automatically acquire the unique configuration information as the target configuration information.

By way of example, after the medical device is connected to the network, the medical device may acquire the target configuration information by receiving and presenting the configuration information automatically recommended by the terminal device and/or the remote control center. By way of example, the configuration information may be recommended to the medical device according to one or more of the device ID of the medical device, the department in which the medical device is used, the ward number, the hospital bed number, and the location information of the medical device, etc. By way of example, if the received configuration information that is automatically recommended is a plurality of configuration information, the medical device 10 may acquire one of recommended configuration information as the target configuration information according to the name and the version number of the configuration information. By way of example, if the received configuration information that is automatically recommended is unique configuration information, the medical device 10 may automatically acquire the unique configuration information as the target configuration information.

By way of example, when the terminal device is a configured medical device and is directly connected to the medical device, the medical device may acquire the configuration information directly from the configured medical device as the target configuration information.

By way of example, the medical device may also be directly connected to the remote control center to acquire the configuration information from the remote control center as the target configuration information. By way of example, the medical device may also access the remote control center via the network to acquire the configuration information from the remote control center as the target configuration information. By way of example, when the remote control center has unique configuration information, the medical device 10 may automatically acquire the unique configuration information as the target configuration information. By way of example, when the remote control center has a plurality of pieces of configuration information, the medical device 10 may acquire (e.g., selected by the user) one of the plurality of the configuration information as the target configuration information. By way of example, the medical device 10 may acquire the configuration information (e.g., selected by the user) as the target configuration information according to one or more of the device ID, the configuration information name, the user ID, the department, the ward number, the hospital bed number, and the location information of the medical device. It may be understood that selection as required may be made in acquiring the configuration information according to one or more of the above items. In an embodiment, when explicitly knowing the device having the configuration that the user desires, the user may directly select the ID of the device to synchronize the configuration information. Alternatively, when explicitly knowing the configuration information name that the user desires, the user may make selection directly according to the configuration information name to obtain the configuration information that the user desires to acquire.

By way of example, after the medical device 10 is connected to the network and the user logs in the medical device, the medical device may automatically acquire the configuration information associated with the user ID of the user via the network to acquire the target configuration information therefrom. By way of example, if the acquired configuration information associated with the user ID of the user include a plurality of configuration information, the medical device 10 may acquire (e.g., selected by the user) one of the plurality of the configuration information as the target configuration information according to the configuration information name, the version of the configuration information, or the storage date of the configuration information, etc. By way of example, when the acquired configuration information associated with the user ID of the user is unique configuration information, the medical device 10 may automatically acquire the unique configuration information as the target configuration information.

By way of example, after being connected to the network, the medical device 10 is capable of automatically acquiring a latest version of the configuration information in the configuration information that corresponds to the location information of the medical device as the target configuration information. Specifically, when the acquired configuration information corresponding to the location information of the medical device is unique configuration information, the medical device 10 may directly acquire the unique configuration information as the target configuration information. When the acquired configuration information corresponding to the location information of the medical device include a plurality of configuration information, the medical device 10 may acquire the latest version of the configuration information in the plurality of the configuration information as the target configuration information.

After acquiring the target configuration information, the medical device 10 sets its configurable options according to the acquired target configuration information, thereby synchronizing the configuration information of the medical device to be the same as that of other medical devices.

It may be understood that during selection, the user may decide how to select the configuration information according to a scenario in which the medical device 10 is used (e.g., the department or the ward). In an embodiment, the medical device 10 may also obtain recommended and downloadable configuration information on the basis of model name information and/or current machine version information.

It may be understood that the configured medical device may also upload the configured target configuration information to the terminal device and/or the remote control center. The uploaded information may be selected by the user, or the configuration information that must be uploaded may be set by default. The uploaded configuration information is stored in the terminal device and/or the remote control center for searching and acquiring by other medical devices.

The medical device of the embodiment of the disclosure is capable of acquiring the required configuration information from the terminal device and/or the remote control center to configure itself according to the acquired configuration information without repeated import and export operations, and thus the operation process is simple and easy, the degree of automation is high, and the time expenditure is less. In addition, automatic configuration and synchronization of the configuration information of the medical device may be implemented across hospitals or across regions. For example, after the user sets the configuration information for a medical device or sets the configuration information on the terminal device and/or the remote control center at a first location, a medical device at a second location may also acquire the configuration information directly from the terminal device and/or the remote control center for installation and synchronization. This way makes it easier for the user to install and configure. Further, when a new medical device needs to be configured or the current medical device needs to be maintained or replaced, the current configuration information can be quickly sent to the medical device, and the configuration information for the new medical device can be simply and quickly configured or the configuration information of the current medical device can be recovered.

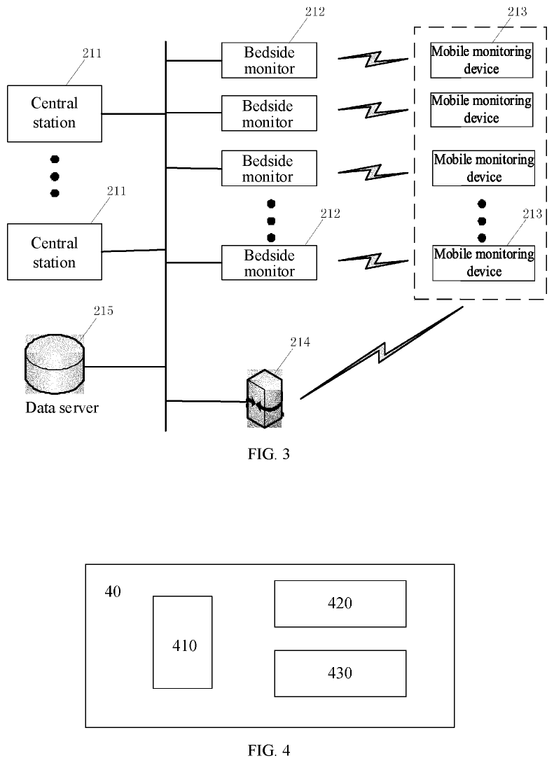

Another embodiment of this disclosure provides a monitor networking system. Referring to FIG. 3, FIG. 3 shows the monitor networking system 20 for use in a hospital in accordance with an embodiment of the disclosure. As shown in FIG. 3, by using the monitor networking system 20, the data of a monitor may be saved as a whole, where patient information and nursing information can be managed centrally and may be stored in association, which facilitates the storage of historical data and associated alarming. In the system shown in FIG. 3, a bedside monitor 212 may be provided for each hospital bed. The bedside monitor 212 may be the above multi-parameter monitor or plug-in monitor. In addition, each bedside monitor 212 may further be paired with a mobile monitoring device 213 for transmission. The mobile monitoring device 213 provides a simple and portable multi-parameter monitor or module assembly. With the wired or wireless communication between the mobile monitoring device 213 and the bedside monitor 212, physiological data generated by the mobile monitoring device may be transmitted to the bedside monitor 212 for display, or transmitted through the bedside monitor 212 to a central station 211 for a doctor or nurse to check, or transmitted through the bedside monitor 212 to a data server 215 for storage. In addition, the mobile monitoring device 213 may further directly transmit, through a wireless network node 214 arranged in the hospital, the physiological data generated by the mobile monitoring to the central station 211 for storage and display, or transmit, through the wireless network node 214 arranged in the hospital, the physiological data generated by the mobile monitoring to the data server 215 for storage. It can be seen that the data corresponding to the physiological parameter displayed on the bedside monitor 212 may originate from the sensor accessory directly connected to the monitor, or from the mobile monitoring device 213, or from the data server. The mobile monitoring device 213 includes a portable monitoring device worn on the body of a patient for mobile monitoring of the patient, and can further include a vital sign monitoring device or a ward round monitoring device having the above-mentioned three working modes, which may be held by the user with hand or moved by a cart or other tools.

It may be understood that the wireless network node 214 may include a router, a switch, a mobile access point, a network bridge, a hub, a storage device, a stand-alone server device, a sensor, a desktop computer, a notebook computer, a mobile phone, as well as other types of network devices.

The bedside monitor 212 and/or the mobile monitoring device 213 may each be the medical device in accordance with first embodiment.

A third embodiment provides a terminal device and/or a remote control center for supplying configuration information to a medical device. By way of example, the terminal device may be a medical device or a mobile storage device, etc. By way of example, the medical device may be a monitor (e.g., a multi-parameter monitor), a ventilator, etc., which is not limited by the disclosure. By way of example, the remote control center may include a server, a central station or a device management system, etc.

Referring to FIG. 4, the terminal device and/or the remote control center 40 may include a memory 410 to store at least one configuration information for the medical device.

By way of example, the at least one configuration information may be set by the user and then stored in the remote control center, or the user may store the configuration information adopted by the configured medical device in the remote control center, or the configured medical device may automatically upload the configuration information adopted by itself to the remote control center, or the at least one configuration information may be the configuration information locally stored and adopted by the medical device, etc. By way of example, the at least one configuration information may be stored in the form of a configuration information packet, etc. By way of example, the at least one configuration information may be identified by one or more of a device ID, a configuration information name, a user ID, a department, a ward number, a hospital bed number, and location information of the medical device.

The terminal device and/or the remote control center 40 can supply one of the at least one configuration information to the medical device as the target configuration information, and the medical device configures a corresponding configurable option according to the target configuration information so as to synchronize its configuration information as the target configuration information.

By way of example, the target configuration information may include at least one of communication authority configuration information, medical parameter configuration information, display configuration information, etc. The medical parameter configuration information refers to configuration information of parameters required by the medical device in the process of completing each medical function, including algorithm configuration information and/or function sequence configuration information, etc. The algorithm configuration information may include an algorithm required to implement a certain medical function, such as algorithms to implement blood pressure measurement, blood oxygen measurement, body temperature measurement, EWS (Early Warning Scoring), a physiological parameter averaging algorithm, etc. The function sequence configuration information may include information of sequence and interval time of executing the algorithms or each medical function. For example, body temperature measurement is performed first, followed by blood pressure measurement second, and finally blood oxygen measurement is performed. As another example, the blood pressure is measured and is to be measured again after 5 minutes. The communication authority configuration information refers to the information that the medical device is connected to an internal network or a medical system of the hospital, which is generally implemented by means of an encryption certificate, such as WiFi encryption certificate (enterprise encryption certificate) and ssl encryption certificate. The display configuration information may include display area information and display style information, where the display area information may be information for limiting the configuration of different measurement functions in each area of a display interface. For example, a blood pressure measurement function area is arranged in an upper right position for prompting blood pressure measurement, inputting and displaying a blood pressure measurement result, and outputting all information related to a blood pressure measurement function, such as a blood pressure alarm. For example, a body temperature measurement value is shown in a lower left corner for prompting body temperature measurement, inputting and displaying a body temperature measurement result, and outputting all information related to a body temperature measurement function, such as a body temperature alarm. The display style information may include information for setting the font, the word size, the word color, the underline, the highlight, etc. of the displayed information. It should be understood that the target configuration information may also include other configuration information, which is not limited by the disclosure.

By way of example, the terminal device and/or the remote control center 40 is capable of broadcasting the at least one configuration information via the network to supply the target configuration information to the medical device. In an embodiment, the medical device may receive, after connecting to the network, these configuration information and acquire the target configuration information from these configuration information. By way of example, when the medical device receives a plurality of configuration information, the medical device may acquire (e.g., selected by the user) one of the plurality of configuration information as the target configuration information according to one or more of the device ID, the configuration information name, the user ID, the department, the ward number, the hospital bed number, and location information of the medical device. By way of example, when the medical device receives unique configuration information, the medical device may automatically acquire the unique configuration information as the target configuration information.

By way of example, after the medical device is connected to the network, the terminal device and/or the remote control center 40 is capable of automatically recommending configuration information to the medical device such that the medical device acquires the target configuration information from the recommended configuration information. By way of example, the terminal device and/or the remote control center 40 may recommend the configuration information to the medical device according to one or more of the device ID of the medical device, the department in which the medical device is used, the ward number, the hospital bed number, the location information of the medical device, etc. Further, when the terminal device and/or the remote control center 40 recommend(s) a plurality of configuration information, the medical device may acquire (e.g., selected by the user) one of the recommended configuration information as the target configuration information according to the name and the version of the configuration information. By way of example, if the terminal device and/or the remote control center 40 recommends unique configuration information, the medical device may automatically acquire the unique configuration information as the target configuration information.

By way of example, when the terminal device is a configured medical device and the configured medical device is directly connected to the medical device, the configured medical device is capable of directly transmitting its configuration information to the medical device as the target configuration information.

By way of example, the remote control center may further include a display device 420 which is configured to, when the remote control center is accessed by the medical device, present at least one configuration information for the medical device to acquire the target configuration information from the at least one configuration information. By way of example, when the remote control center has a plurality of configuration information, the medical device may acquire (e.g., selected by the user) one of the plurality of configuration information as the target configuration information according to one or more of the device ID, the configuration information name, the user ID, the department, the ward number, the hospital bed number, and location information of the medical device. By way of example, when the remote control center has unique configuration information, the medical device may automatically acquire the unique configuration information as the target configuration information.

By way of example, after the medical device is connected to the network and the user logs in the medical device, the terminal device and/or the remote control center may automatically transmit the configuration information associated with the user ID of the user via the network to the medical device such that the medical device acquires the target configuration information. By way of example, when the configuration information transmitted by the terminal device and/or the remote control center and associated with the user ID of the user include a plurality of configuration information, the medical device may acquire the configuration information (e.g., selected by the user) as the target configuration information according to the configuration information name, the version of the configuration information, and/or the storage date of the configuration information, etc. By way of example, when the configuration information transmitted by the terminal device and/or the remote control center and associated with the user ID of the user is unique configuration information, the medical device may automatically acquire the unique configuration information as the target configuration information.

By way of example, the terminal device and/or the remote control center 40 may further include a detection apparatus 430 capable of automatically detecting the location information where the medical device is located after the medical device is connected to the network. Further, when the detection apparatus 430 detects the location information where the medical device is located, the terminal device and/or the remote control center is capable of automatically transmitting a latest version of the configuration information in the configuration information that corresponds to the location information to the medical device as the target configuration information. For example, when the terminal device and/or the remote control center has unique configuration information corresponding to the location information of the medical device, the unique configuration information may be automatically and directly transmitted to the medical device as the target configuration information. When the terminal device and/or the remote control center has a plurality of configuration information corresponding to the location information of the medical device, the latest version of the configuration information in the configuration information that corresponds to the location information may be automatically transmitted to the medical device as the target configuration information.

The configuration information is saved in the terminal device and/or the remote control center. Further, the terminal device and/or the remote control center counts and classifies the configuration information stored in the terminal device and/or the remote control center by means of big data learning. In an embodiment, the terminal device and/or the remote control center counts and classifies the configuration information used by each medical device according to a scenario in which the medical device is used, such as a department or a ward, so as to find the configuration information used frequently by users in different departments or wards, determine the scenario in which the medical device is used according to the location information, and send the configuration information with high frequency of use to the medical device.

The terminal device and/or the remote control center of the embodiment of the disclosure is capable of supplying the configuration information to the medical device without repeated import and export operations, and thus the operation process is simple and easy, the degree of automation is high, and the time expenditure is less. In addition, automatic configuration and synchronization of the configuration information for the medical device may be implemented across hospitals or across regions. For example, after the user sets the configuration information for a medical device or sets the configuration information on the terminal device and/or the remote control center at a first location, a medical device at a second location may also acquire the configuration information directly from the terminal device and/or the remote control center for installation and synchronization. This way makes it easier for the user to install and configure. Further, when a new medical device needs to be configured or the current medical device needs to be maintained or replaced, the current configuration information can be quickly sent to the medical device, and the configuration information for the new medical device can be simply and quickly configured or the configuration information of the current medical device can be recovered.

A fourth embodiment provides a method 50 for synchronizing configuration information of a medical device. By way of example, the medical device may be a monitor (e.g., a multi-parameter monitor), a ventilator, etc., which is not limited by the disclosure.

Referring to FIG. 5, FIG. 5 shows a method 500 for synchronizing configuration information of a medical device in accordance with an embodiment of the disclosure. By way of example, the method 500 may include the following steps S510-S520.

At step S510, the configuration information is acquired from a terminal device and/or a remote control center as target configuration information, where the target configuration information includes at least one of communication authority configuration information, medical parameter configuration information and display configuration information, and the terminal device and/or the remote control center has at least one configuration information.

The target configuration information may be the same configuration information as the configuration information of a medical device that has been set already, or may be any configuration information that the user desires.

The medical parameter configuration information refers to configuration information of parameters required by the medical device in the process of completing each medical function, including algorithm configuration information and/or function sequence configuration information, etc. The algorithm configuration information may include an algorithm required to implement a certain medical function, such as algorithms to implement blood pressure measurement, blood oxygen measurement, body temperature measurement, EWS (Early Warning Scoring), a physiological parameter averaging algorithm, etc. The function sequence configuration information may include information of sequence and interval time of executing the algorithms or each medical function. For example, body temperature measurement is performed first, followed by blood pressure measurement second, and finally blood oxygen measurement is performed. As another example, the blood pressure is measured and is to be measured again after 5 minutes. The communication authority configuration information refers to the information that the medical device is connected to an internal network or a medical system of the hospital, which is generally implemented by means of an encryption certificate, such as WiFi encryption certificate (enterprise encryption certificate) and ssl encryption certificate. The display configuration information may include display area information and display style information, where the display area information may be information for limiting the configuration of different measurement functions in each area of a display interface. For example, a blood pressure measurement function area is arranged in an upper right position for prompting blood pressure measurement, inputting and displaying a blood pressure measurement result, and outputting all information related to a blood pressure measurement function, such as a blood pressure alarm. For example, a body temperature measurement value is shown in a lower left corner for prompting body temperature measurement, inputting and displaying a body temperature measurement result, and outputting all information related to a body temperature measurement function, such as a body temperature alarm. The display style information may include information for setting the font, the word size, the word color, the underline, the highlight, etc. of displayed information. It should be understood that the target configuration information may also include other configuration information, which is not limited by the disclosure.

By way of example, the terminal device may be a medical device or a mobile storage device, etc. By way of example, the remote control center may include a server, a central station, a device management system, etc. By way of example, the at least one configuration information may be set by the user and then stored in the terminal device and/or the remote control center, or the user may store the configuration information adopted by the configured medical device in the terminal device and/or the remote control center, or the medical device may automatically upload the configuration information adopted by itself to the remote control center, or the at least one configuration information may be configuration information locally stored and adopted by the medical device, etc. By way of example, the at least one configuration information may be stored in the form of a configuration information packet, etc. By way of example, the at least one configuration information may be identified by one or more of a device ID, a configuration information name, a user ID, a department, a ward number, a hospital bed number, and location information of the medical device.

By way of example, the medical device can acquire the target configuration information from the terminal device and/or the remote control center by: receiving the configuration information that the terminal device and/or the remote control center has and that is broadcast by the terminal device and/or the remote control center via a network, and acquiring the target configuration information from the received configuration information. In an embodiment, the terminal device and/or the remote control center broadcasts, via the network, the configuration information that the terminal device and/or the remote control center has, and the medical device receives the broadcast configuration information after connecting to the network and acquires the configuration information required by the medical device as the target configuration information. By way of example, if the received configuration information include a plurality of configuration information, the medical device may acquire (e.g., selected by the user) one of the plurality of configuration information as the target configuration information. By way of example, the medical device may acquire the configuration information (e.g., selected by the user) as the target configuration information according to one or more of the device ID, the configuration information name, the user ID, the department, the ward number, the hospital bed number, and the location information of the medical device. By way of example, if the received configuration information is unique configuration information, the medical device may automatically acquire the unique configuration information as the target configuration information.

By way of example, the medical device may acquire the target configuration information from the terminal device and/or the remote control center by: receiving and presenting the configuration information automatically recommended by the terminal device and/or the remote control center when connecting to the network, so as to acquire the target configuration information from the recommended configuration information. By way of example, the medical device may acquire the target configuration information therefrom according to one or more of the device ID of the medical device, the department in which the medical device is used, the ward number, the hospital bed number, and the location information of the medical device. By way of example, when the received configuration information that is automatically recommended includes a plurality of configuration information, the medical device may acquire a desired one (e.g., selected by the user) of the plurality of the recommended configuration information as the target configuration information according to the name and the version of the configuration information. By way of example, when the received configuration information that is automatically recommended is unique configuration information, the medical device may automatically acquire the unique configuration information as the target configuration information.

By way of example, when the terminal device is a configured medical device and the medical device is directly connected to the configured medical device, the medical device is capable of acquiring its configuration information directly from the configured medical device as the target configuration information.

By way of example, the medical device is alternatively directly connected to the remote control center to acquire the configuration information as the target configuration information. By way of example, the medical device may access the remote control center via the network, and acquire the configuration information from the remote control center as the target configuration information. By way of example, when the remote control center has a plurality of configuration information, the medical device may acquire (e.g., selected by the user) one of the plurality of configuration information as the target configuration information. By way of example, the medical device may acquire the configuration information (e.g., selected by the user) as the target configuration information according to any one of the device ID, the configuration information name, the user ID, the department, the ward number, the hospital bed number, and the location information of the medical device. By way of example, when the remote control center has unique configuration information, the medical device may automatically acquire the unique configuration information as the target configuration information.

By way of example, the method 500 may further include: after the medical device is connected to the network and the user logs in the medical device, the configuration information associated with the user ID of the user is automatically acquired via the network to acquire the target configuration information therefrom. By way of example, when the acquired configuration information associated with the user ID of the user includes a plurality of configuration information, the medical device may acquire the target configuration information (e.g., selected by the user) according to the configuration information name, the version of the configuration information, and/or the storage date of the configuration information, etc. By way of example, when the acquired configuration information associated with the user ID of the user is unique configuration information, the medical device may automatically acquire the unique configuration information as the target configuration information.

By way of example, after being connected to the network, the medical device is capable of automatically acquiring a latest version of the configuration information in the configuration information that corresponds to the location information of the medical device as the target configuration information. Specifically, when the acquired configuration information corresponding to the location information of the medical device is unique configuration information, the unique configuration information may be directly acquired as the target configuration information. When the acquired configuration information corresponding to the location information of the medical device includes a plurality of configuration information, the latest version of the configuration information in the plurality of configuration information may be acquired as the target configuration information.

At step S520, a corresponding configurable option is set for the medical device according to the target configuration information so as to synchronize the configuration information of the medical device as the target configuration information.

By way of example, the configurable option may include a communication authority configuration item, a medical parameter configuration item, and/or a display configuration item, etc., which is not limited by the disclosure. The configurable option may be set according to the target configuration information to synchronize the configuration information of the medical device as the target configuration information.

The method for synchronizing the configuration information for the medical device of the embodiment of the disclosure does not need to repeat import and export operations, the operation process is simple and easy, the degree of automation is high, and the time expenditure is less.

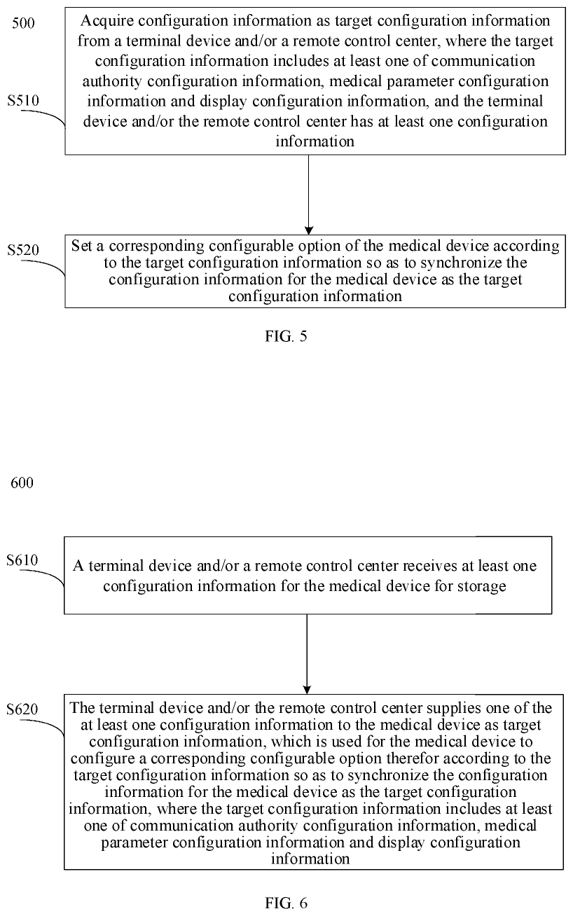

A fifth embodiment provides a method for providing configuration information for a medical device. Referring to FIG. 6, FIG. 6 shows a method 600 for providing configuration information for a medical device in accordance with an embodiment of the disclosure. As shown in FIG. 6, the method 600 may include the following steps 610-620.

At step 610, a terminal device and/or a remote control center receive(s) at least one configuration information for the medical device for storage.

By way of example, the medical device may be a monitor (e.g., a multi-parameter monitor), and/or a ventilator, etc., which is not limited by the disclosure. By way of example, the terminal device may be a medical device, and/or a mobile storage device, etc. By way of example, the remote control center may include a server, a central station, and/or a device management system, etc.

By way of example, the terminal device and/or the remote control center may receive the configuration information set by the user for storage, or may receive the configuration information adopted by a configured medical device for storage, or may receive the configuration information adopted and automatically uploaded by the medical device for storage, or the configuration information locally stored and adopted by the medical device, etc. By way of example, the terminal device and/or the remote control center may store the at least one configuration information in the form of a configuration information packet, etc. By way of example, the at least one configuration information may be identified by one or more of a device ID, a configuration information name, a user ID, a department, a ward number, a hospital bed number, and location information of the medical device.

At step 620, the terminal device and/or the remote control center may supply one of the at least one configuration information to the medical device as the target configuration information, and the medical device configures a corresponding configurable option thereof according to the target configuration information so as to synchronize its configuration information as the target configuration information, where the target configuration information includes at least one of communication authority configuration information, medical parameter configuration information and display configuration information.

By way of example, the configurable option may include a communication authority configuration item, a medical parameter configuration item, and/or a display configuration item, etc., which is not limited by the disclosure. The configurable option may be set according to the target configuration information to synchronize the configuration information of the medical device as the target configuration information.

By way of example, the target configuration information may be the same configuration information as the configuration information of the medical device that has been set already, or may be any configuration information that the user desires.

By way of example, the target configuration information may include at least one of communication authority configuration information, medical parameter configuration information, display configuration information, etc. The medical parameter configuration information refers to configuration information of parameters required by the medical device in the process of completing each medical function, including algorithm configuration information, function sequence configuration information, etc. The algorithm configuration information may include an algorithm required to implement a certain medical function, such as algorithms to implement blood pressure measurement, blood oxygen measurement, body temperature measurement, EWS (Early Warning Scoring), a physiological parameter averaging algorithm, etc. The function sequence configuration information may include information of sequence and interval time of executing the algorithms or each medical function. For example, body temperature measurement is performed first, followed by blood pressure measurement second, and finally blood oxygen measurement is performed. As another example, the blood pressure is measured and is to be measured again after 5 minutes. The communication authority configuration information refers to the information that the medical device is connected to an internal network or a medical system of the hospital, which is generally implemented by means of an encryption certificate, such as WiFi encryption certificate (enterprise encryption certificate) and ssl encryption certificate. The display configuration information may include display area information and display style information, where the display area information may be information for limiting the configuration of different measurement functions in each area of a display interface. For example, a blood pressure measurement function area is arranged in an upper right position for prompting blood pressure measurement, inputting and displaying a blood pressure measurement result, and outputting all information related to a blood pressure measurement function, such as a blood pressure alarm. For example, a body temperature measurement value is shown in a lower left corner for prompting body temperature measurement, inputting and displaying a body temperature measurement result, and outputting all information related to a body temperature measurement function, such as a body temperature alarm. The display style information may include information for setting the font, the word size, the word color, the underline, the highlight, etc. of the displayed information. It should be understood that the target configuration information may also include other configuration information, which is not limited by the disclosure.

By way of example, the terminal device and/or the remote control center broadcast(s), via a network, the configuration information that the terminal device and/or the remote control center has, such that the medical device receives and acquires the target configuration information from the broadcast configuration information.

By way of example, when it is detected that the medical device is connected to the network, the terminal device and/or the remote control center automatically recommend the configuration information to the medical device such that the medical device acquires the target configuration information therefrom. By way of example, the terminal device and/or the remote control center may recommend the configuration information to the medical device according to one or more of the device ID of the medical device, the department in which the medical device is used, the ward number, the hospital bed number, and the location information of the medical device, etc. By way of example, when the automatically recommended configuration information that is received by the medical device includes a plurality of configuration information, the medical device may acquire the desired configuration information (e.g., selected by the user) from the recommended configuration information as the target configuration information according to the name and the version of the configuration information. By way of example, when the automatically recommended configuration information that is received by the medical device is unique configuration information, the medical device may automatically acquire the unique configuration information as the target configuration information.

By way of example, the method 600 may further include: directly transmitting the configuration information of a configured medical device to the medical device as the target configuration information when the configured medical device is directly connected to the medical device.

By way of example, the remote control center presents, when assessed by the medical device, the configuration information for the medical device to acquire the target configuration information therefrom. By way of example, when the remote control center has a plurality of configuration information, the medical device may acquire (e.g., selected by the user) one of the plurality of configuration information as the target configuration information. By way of example, the medical device may acquire the configuration information (e.g., selected by the user) as the target configuration information according to one or more of the device ID, the configuration information name, the user ID, the department, the ward number, the hospital bed number, and the location information of the medical device. By way of example, when the remote control center has unique configuration information, the medical device may automatically acquire the unique configuration information as the target configuration information.

By way of example, after the medical device is connected to the network, the location information where the medical device is located is automatically detected, and a latest version of the configuration information in the configuration information corresponding to the location information is automatically transmitted to the medical device as the target configuration information. Specifically, when the transmitted configuration information corresponding to the location information of the medical device is unique configuration information, the medical device may directly acquire the unique configuration information as the target configuration information. When the transmitted configuration information corresponding to the location information of the medical device include a plurality of configuration information, the medical device may acquire the latest version of configuration information of the plurality of configuration information as the target configuration information.

The method for supplying the configuration information for the medical device of the embodiment of the disclosure does not need to repeat import and export operations, the operation process is simple and easy, the degree of automation is high, and the time expenditure is short.

Another embodiment provides a computer-readable medium which stores a computer program thereon, which, when running, causes the method as described in the above embodiments to be carried out. Any tangible, non-transitory computer-readable medium may be used, including magnetic storage devices (hard disks, floppy disks, etc.), optical storage devices (CD-ROM, DVD, Blue-ray discs, etc.), flash memories, and/or the like. These computer program instructions may be loaded to a general-purpose computer, a dedicated computer, or other programmable data processing device to form a machine, such that these instructions executed on a computer or other programmable data processing device may generate an apparatus that implements a specified function. These computer program instructions can also be stored in a computer-readable memory that can instruct a computer or other programmable data processing device to operate in a specific manner, such that the instructions stored in the computer-readable memory can form a manufactured product, including an implementation apparatus that implements a specified function. The computer program instructions may also be loaded onto a computer or other programmable data processing device, such that a series of operation steps are executed on the computer or other programmable device to produce a computer-implemented process, such that the instructions executed on the computer or other programmable device may provide steps for implementing a specified function.

While example embodiments have been described herein with reference to the accompanying drawings, it should be understood that the above example embodiments are merely illustrative and are not intended to limit the scope of the disclosure thereto. Those of ordinary skill in the art may make various changes and modifications therein without departing from the scope and spirit of the disclosure. All such changes and modifications are intended to be included in the scope of the disclosure as claimed in the appended claims.

A large number of specific details are explained in this specification provided herein. However, it can be understood that the embodiments of the disclosure can be practiced without these specific details. In some examples, well-known methods, structures, and technologies are not shown in detail, so as not to obscure the understanding of this specification.

Similarly, it should be understood that in order to simplify the disclosure and help to understand one or more of various aspects of the disclosure, in the description of the exemplary embodiments of the disclosure, various features of the disclosure are sometimes together grouped into an individual embodiment, figure or description thereof. However, the method of the disclosure should not be construed as reflecting the following intention: namely, the disclosure set forth requires more features than those explicitly stated in each claim. More precisely, as reflected by the corresponding claims, the inventive point thereof lies in that features that are fewer than all the features of an individual embodiment disclosed may be used to solve the corresponding technical problem. Therefore, the claims in accordance with the particular embodiments are thereby explicitly incorporated into the particular embodiments, wherein each claim itself serves as an individual embodiment of the disclosure.

Those skilled in the art should understand that, in addition to the case where features are mutually exclusive, any combination may be used to combine all the features disclosed in this specification (along with the appended claims, abstract, and drawings) and all the processes or units of any of methods or devices as disclosed. Unless explicitly stated otherwise, each feature disclosed in this specification (along with the appended claims, abstract, and drawings) may be replaced by an alternative feature that provides the same, equivalent, or similar object.

Furthermore, those skilled in the art should understand that although some of the embodiments described herein include some but not other features included in other embodiments, combinations of features of different embodiments are meant to be within the scope of the disclosure, and form different embodiments. For example, in the claims, any one of the embodiments set forth thereby can be used in any combination.

It should be noted that the description of the disclosure made in the above-mentioned embodiments is not to limit the disclosure, and those skilled in the art may design alternative embodiments without departing from the scope of the appended claims. In the claims, any reference signs placed between parentheses should not be construed as limitation on the claims. The term “comprising” does not exclude the presence of elements or steps not listed in the claims. The term “a” or “an” preceding an element does not exclude the presence of a plurality of such elements. The disclosure may be implemented by means of hardware including several different elements and by means of an appropriately programmed computer. In unit claims listing several apparatuses, several of these apparatuses may be specifically embodied by one and the same item of hardware. The use of the terms “first”, “second”, “third”, etc. does not indicate any order. These terms may be interpreted as names.

The above descriptions are merely the specific embodiments of the disclosure or the description of the specific embodiments, but the scope of protection of the disclosure is not limited thereto. Changes or substitutions which may be readily contemplated by those skilled in the art within the technical scope disclosed in the disclosure fall within the scope of protection of the disclosure. The scope of protection of the disclosure should be subject to the scope of protection of the claims.

What is claimed is:

1. A method for synchronizing configuration information of a medical device, comprising:
acquiring configuration information from at least one of a terminal device and a remote control center as target configuration information, wherein the target configuration information comprises at least one of communication authority configuration information, medical parameter configuration information and display configuration information, and at least one of the terminal device and the remote control center has at least one configuration information; and
setting a corresponding configurable option of the medical device according to the target configuration information so as to synchronize the configuration information of the medical device as the target configuration information.

2. The method of claim 1, wherein acquiring configuration information from at least one of a terminal device and a remote control center as target configuration information comprises:
receiving the configuration information that is broadcast by at least one of the terminal device and the remote control center via a network; and
acquiring the target configuration information from the received configuration information.

3. The method of claim 2, wherein acquiring the target configuration information from the received configuration information comprises:
when the received configuration information comprises a plurality of configuration information, acquiring one of the plurality of the received configuration information as the target configuration information; and
when the received configuration information is unique configuration information, automatically acquiring the unique configuration information as the target configuration information.

4. The method of claim 1, wherein acquiring configuration information from at least one of a terminal device and a remote control center as target configuration information comprises:
receiving and presenting, when the medical device is connected to a network, the configuration information automatically recommended by at least one of the terminal device and the remote control center so as to acquire the target configuration information.

5. The method of claim 1, wherein acquiring configuration information from at least one of a terminal device and a remote control center as target configuration information comprises:
when the terminal device is a configured medical device and the medical device is directly connected to the configured medical device, acquiring the configuration information directly from the configured medical device as the target configuration information.

6. The method of claim 1, wherein acquiring configuration information from at least one of a terminal device and a remote control center as target configuration information comprises:
accessing the remote control center via a network, and acquiring the target configuration information from the remote control center.

7. The method of claim 6, wherein acquiring the target configuration information from the remote control center, comprises:
when the remote control center has a plurality of configuration information, acquiring one of the plurality of the configuration information as the target configuration information; and
when the remote control center has unique configuration information, automatically acquiring the unique configuration information as the target configuration information.

8. The method of claim 1, wherein the at least one configuration information is identified by one or more of a device ID, a configuration information name, a user ID, a department, a ward number, a hospital bed number, and location information of the medical device, and the method further comprises: acquiring the target configuration information from the at least one configuration information according to one or more of the device ID, the configuration information name, the user ID, the department, the ward number, the hospital bed number, and the location information of the medical device.

9. The method of claim 8, further comprising:
automatically acquiring, after the medical device is connected to the network, a latest version of configuration information in the configuration information that corresponds to the location information of the medical device as the target configuration information.

10. A medical device with configuration information to be synchronized, comprising at least one processor, the medical device is set with a configured option and a configurable option,
wherein the at least one processor is configured to set the configurable option according to target configuration information to synchronize the configuration information of the medical device as the target configuration information, and
the target configuration information is configuration information that is acquired by the medical device from at least one of a terminal device and a remote control center and is comprised of at least one of communication authority configuration information, medical parameter configuration information and display configuration information, and at least one of the terminal device and the remote control center has at least one configuration information.

11. The medical device of claim 10, wherein the target configuration information is acquired from the configuration information received by means of network broadcast from at least one of the terminal device and the remote control center.

12. The medical device of claim 11, wherein when the received configuration information comprises a plurality of configuration information, the medical device acquires one of the plurality of the configuration information as the target configuration information; and when the received configuration information is unique configuration information, the medical device automatically acquires the unique configuration information as the target configuration information.

13. The medical device of claim 10, wherein when the medical device is connected to a network, the medical device is capable of receiving and presenting the configuration information automatically recommended by at least one of the terminal device and the remote control center so as to acquire the target configuration information.

14. The medical device of claim 10, wherein when the terminal device is a configured medical device and the medical device is directly connected to the configured medical device, the medical device is capable of directly acquiring the configuration information of the configured medical device as the target configuration information.

15. The medical device of claim 10, wherein the medical device is capable of acquiring, when accessing the remote control center via a network, the target configuration information from the remote control center.

16. The medical device of claim 15, wherein when the remote control center has a plurality of configuration information, the medical device acquires one of the plurality of configuration information as the target configuration information; and when the remote control center has unique configuration information, the medical device automatically acquires the unique configuration information as the target configuration information.

17. The medical device of claim 10, wherein the at least one configuration information is identified by one or more of a device ID, a configuration information name, a user ID, a department, a ward number, a hospital bed number and location information of the medical device, the medical device is capable of acquiring one of the at least one configuration information as the target configuration information according to one or more of the device ID, the configuration information name, the user ID, the department, the ward number, the hospital bed number, and the location information of the medical device.

18. The medical device of claim 17, wherein the medical device is capable of automatically acquiring the configuration information associated with the user ID of a user via a network to acquire the target configuration information therefrom.

19. The medical device of claim 18, wherein when the acquired configuration information associated with the user ID of the user is unique configuration information, the medical device is capable of automatically acquiring the unique configuration information as the target configuration information.

20. The medical device of claim 17, wherein the medical device is capable of automatically acquiring, after being connected to a network, a latest version of configuration information in the configuration information that corresponds to the location information of the medical device as the target configuration information.
